(19) United States
(12) Patent Application Publication
(10) Pub. No.: US 2023/0233180 A1
(43) Pub. Date: Jul. 27, 2023

(54) Emergency Ultrasound Gel

(71) Applicant: Mark Edward Fenzl, Sarasota, FL (US)

(72) Inventor: Mark Edward Fenzl, Sarasota, FL (US)

(21) Appl. No.: 17/885,572

(22) Filed: Aug. 11, 2022

Related U.S. Application Data
(60) Provisional application No. 63/361,819, filed on Jan. 21, 2022.

(51) Int. Cl. A61B 8/00; C08J 3/075; C08K 5/06; C08K 5/17; C08K 11/00

(52) U.S. Cl. CPC A61B 8/4281; C08J 3/075; C08K 5/06; C08K 5/175; C08K 11/00; C08J 2300/14

(57) ABSTRACT

The primary objective of the present invention is to provide an ultrasound gel that can be applied directly to wounds for emergency personnel in the field or in the Emergency Department. The problems is that one does not want to contaminate a wound with normal ultrasound gel and sterile ultrasound gel may take extra time to apply and cause irritation or sensitization to an open wound. This ultrasound gel is designed to be applied to open contaminated wounds by medical personnel for diagnostic evaluation of trauma, critical care, or emergency patients.

### CROSS-REFERENCE TO RELATED APPLICATIONS

This application claims the benefit of U.S. Provisional Application 63/361,819, filed on Jan. 21, 2022. The above application is incorporated herein in reference to a combination preservative.

### FIELD

The present disclosure relates to a medical product and, more particularly, an ultrasound gel. This ready-to-use ultrasound gel may be applied to open contaminated wounds, has a broad-spectrum bactericidal effect, is broad-spectrum fungistatic, and does not cause irritation or sensitivity to open wounds.

### BACKGROUND

This section provides background information related to the present disclosure which is not necessarily prior art.

The present invention relates to an improved ultrasound gel that is ready-to-use, sterile, antimicrobial, and may be used on open wounds. Ultrasonography is the process of using an ultrasound machine that transmits ultrasound waves for a diagnostic or therapeutic purpose. Ultrasonography requires ultrasound gel to transmit ultrasound waves to and from the transducer on the ultrasound machine to the patient and back.

Throughout the process of ultrasonography, an ultrasonographer may place ultrasound gel on the patient multiple times using ultrasound gel from a bottle or packets. Often ultrasound is used in the trauma bay of an emergency department or critical care floor for diagnostics. For example it may be used to diagnose internal bleeding, hypotension, pneumothorax, pericardial effusion, congestive heart failure, uterine dysfunction, fetal wellness, appendicitis, cholecycstitis, or blood flow to an extremity. It is also used to help with procedures, such as the placement of an IV or central line, which can be critical during an emergency. Ultrasound is being used more often in the field by paramedics and the military. Ultrasound can be a powerful tool in the golden hour of trauma. Early diagnosis can lead to early treatment and if a paramedic in the field can diagnose a problem early, teams in the ER or the hospital may have the appropriate equipment and specialists on hand to provide quick and effective care for an incoming trauma patient.

Sometimes the healthcare provider may have to use ultrasound over an area that has an open wound and does not have sterile ultrasound gel or does not have the time to prepare sterile ultrasound gel. Non-sterile ultrasound gels may not be applied to open wounds without causing contamination and infection. One way to overcome this is to use a ready-to-use broad-spectrum antimicrobial, sterile ultrasound gel that is not irritating and does not cause allergic reaction to open wounds.

There are sterile ultrasound gels on the market. Sterile ultrasound gels on the market are not bactericidal or fungicidal and only come in 20 ml packets. One has to repeatedly open a new packet to obtain more gel while maintaining a sterile field. Performing ultrasound in the field may be difficult. For example, opening ultrasound packets at night or in a moving ambulance, helicopter, or ship may be difficult. In a battlefield situation, there may no opportunity to create a sterile field and early diagnosis is critical for triage and medical management in the field. In this situation having a ready-to-use sterile, bactericidal, fungistatic, non-irritating ultrasound gel is advantageous. Providing a sterile ultrasound gel in a larger dispenser that does not require multiple sachets may facilitate the process of ultrasound in the field, in the emergency room, or in the intensive care unit. On the battlefield, it may save lives.

Sodium hydroxymethylglycinate is a bactericidal preservative that kills a wide range of bacteria at 0.09% and can be used in the Unites States and Europe at a concentration of 1%, but it does not have strong antifungal effect. Phenoxyethanol is synergistic with ethlhexylglycerin, is a fungistatic preservative for a wide variety of yeast and fungi at 0.25%, and is accepted throughout the world at a concentration of 1.1%. Combining these two preservatives creates an ultrasound gel that is bactericidal of a broad spectrum of bacteria, is bacteriostatic of a broad spectrum of yeast and fungi, and does not irritate tissues in open wounds.

There are a variety of essential oils that are fungistatic. Examples include but are not limited to cinnamon essential oil, thyme essential oil, clove essential oil, oregano essential oil, tea tree essential oil, lemongrass essential oil, palmarosa essential oil, eucalyptus essential oils, lavender essential oil, and citrus essential oils. The chemical components of essential oils also may have a natural anesthetic effect so that it not only inhibits microbes, but also decreases pain and may have a fragrance that elevates mood. This combination may have a synergistic effect on stress and blood pressure. This ultrasound gel may contain an anesthetic essential oil or component of an essential oil and may contain a fungistatic or fungicidal essential oil or component of an essential oil.

TABLE 1
Essential oil components with local anesthetic activity.

| Class | Essential Oil Component |
|---|---|
| Acyclic Monoterpinoid | Linalool |
| Acyclic Monoterpinoid | Linalyl acetate |
| Acyclic Monoterpinoid | Geranial |
| Acyclic Monoterpinoid | Neral |
| Acyclic Monoterpinoid | Citronellol |
| Acyclic Monoterpinoid | Citronellal |
| Acyclic Monoterpinoid | Geraniol |
| Acyclic Monoterpinoid | Geranyl acetate |
| Monocyclic Monoterpinoid | Menthol |
| Monocyclic Monoterpinoid | Cymene |
| Monocyclic Monoterpinoid | Thymol |
| Monocyclic Monoterpinoid | Carvacrol |
| Monocyclic Monoterpinoid | Terpineol |
| Monocyclic Monoterpinoid | Terpinene |
| Monocyclic Monoterpinoid | Menthone |
| Monocyclic Monoterpinoid | Carvone |
| Monocyclic Monoterpinoid | Pulegone |
| Monocyclic Monoterpinoid | Limonene |
| Monocyclic Monoterpinoid | Eugenol |
| Bicyclic Monoterpinoid | Eucalyptol |
| Bicyclic Monoterpinoid | Cineol |
| Bicyclic Monoterpinoid | Pinene |
| Bicyclic Monoterpinoid | Fenchone |
| Bicyclic Monoterpinoid | Borneol |
| Bicyclic Monoterpinoid | Bornyl acetate |
| Bicyclic Monoterpinoid | Camphene |
| Bicyclic Monoterpinoid | Cinnamaldehyde |
| Bicyclic Monoterpinoid | Anethole |
| Bicyclic Monoterpinoid | Estragole |
| Sesquiterpenoid | Caryophyllene |

TABLE 2
Minimal Inhibitory Concentration and Minimal Fungicidal Concentration
of essential oils against multiple strains of Candida albicans.

| | Minimal | Minimal |
|---|---|---|
| | Inhibitory | Fungicidal |
| Essential Oils | Concentration by % | Concentration by % |
| Lemongrass oil | 0.06 | 0.12 |
| Clove Leaf Oil | 0.12 | 0.12 |
| Japanese oil | 0.12 | 0.12 |
| Geranium oil | 0.12 | 0.12 |
| Cinnamon oil | 0.01 | 0.03 |
| Motiarosha oil | 0.09 | 0.12 |
| Orange oil | 1 | 2 |
| Ylang-ylang oil | 0.5 | 1 |
| Gingergrass oil | 0.15 | 0.15 |
| Peppermint oil | 0.3 | 0.3 |
| Ocimum oil | 1 | 1 |
| Lemon oil | 1 | 2 |
| Camphor oil | 0.5 | 1 |
| Rosemary oil | 1 | 3 |
| Tulsi oil | 0.25 | 0.5 |
| Bergamot oil | 1 | 2 |
| Tea tree oil | 0.25 | 0.5 |
| Clary Sage oil | 2 | 3 |
| Eucalyptus oil | 1.5 | 2.5 |
| Citronella oil | 1 | 2 |
| Rose oil | 2 | 3 |

Often traumatic wounds are painful and topical anesthetic in the ultrasound gel may reduce pain that may decrease blood pressure and catecholamines that increase risk of exsanguination. Natural or synthetic anesthetics may be added to the product to reduce pain and improve outcome for trauma patients. Some anesthetics also have a fungistatic preservative effect.

TABLE 3
Minimal Fungicidal Concentration of Bupivacaine
and Lidocaine against Candida albicans.

| | MIC | MIC |
|---|---|---|
| | Lidocaine | Bupivacaine |
| Microorganism | mg/ml | mg/ml |
| Candida albicans M36 | 5 | 2.5 |
| Candida albicans H33 | 5 | 2.5 |
| Candida albicans SCO | 2.5 | 1.3 |
| Canddia albicans M9 | 2.5 | 1.3 |
| Candida albicans Ser5 | 5.0 | 2.5 |
| Candida. albicans H65 | 2.5 | 1.3 |
| Candida albicans M28 | 2.5 | 1.3 |
| Candida albicans H38 | 5 | 2.5 |
| Candida albicans 10231 | 5 | 2.5 |
| Candida albicans H37 | 10 | 5 |

Aloe vera inhibits a variety of bacteria and aloe vera extract 1:100 at concentrations of 0.2% to 0.5% increase wound absorption of water. Aloe vera inhibits Candida albicans growth at 10%.

Chelating agents may have a synergistic effect with sodium hydroxymethylglycinate. Chelating agents bind with metal ions so that they are not available to microorganisms. Examples that do not limit this patent include disodium EDTA and citric acid. These may be used in an ultrasound gel.

“Airless” pump dispensers pump out a product without bringing contaminated air into the bottle. This reduces the risk of microbial contamination. Each pump dispenses a quantifiable amount of product. The ultrasound gel may be pumped from an airless pump dispenser container to provide a consistent, measurable amount of ultrasound gel and protect from contamination.

The combination of preservatives has a synergistic effect on killing microbial contaminants. Advantageously, non-limiting examples of use of this product may include an ultrasound gel in an airless container containing fungicidal ingredients.

### SUMMARY OF THE INVENTION

In concordance with the instant disclosure, this invention is an improved sterile, antimicrobial, non-irritating ultrasound gel.

One example of several potential medical products that does not limit this patent application, is an ultrasound gel preserved with a combination of sodium hydroxymethylglycinate and phenoxyethanol/ethylhexylglycerine. This combination is synergistic. Deionized water may be used to create the gel. Carbomer may be used as a gelling agent and glycerol as a humectant. Aloe vera may be used to moisturize the skin. This product may be dispensed from an airless dispenser to reduce the risk of contamination and quantify how much is being used.

A variation that does not limit this patent is an ultrasound gel containing deionized water, aloe vera, benzocaine, carbomer, and sodium hydroxymethylglycinate in an airless container.

A second variation that does not limit this patent is an ultrasound gel containing deionized water, aloe vera, carbomer, sodium hydroxymethylglycinate, and linalool in an airless container.

Other variations may include a natural or synthetic anesthetic, chelating agent, essential oil, metal nanoparticles, monoglyceride, and/or natural fragrance for antimicrobial, anesthetic, or fragrance effect.

### DETAILED DESCRIPTION

### Definitions:

Ultrasound gel—refers to scanning gel or gels, transmission gel or gels, ultrasound gel or gels, ultrasound lotion or lotions, or any other solution designed or sold for the purpose of transmitting ultrasound waves.

Bactericidal—kills bacteria.

Bacteriostatic—inhibits bacterial growth.

Fungistatic—inhibits yeast and fungal growth.

Fungicidal—kills yeast and fungi.

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

The present disclosure relates to an emergency ultrasound gel, that does not limit this patent, comprising vegetable glycerin (5%), phenoxyethanol (0.5%)/ethylhexylglycerin (0.1%), sodium hydroxymethylglycinate (1%), aloe vera extract (0.2%), carbomer (0.7%), and deionized water in quantities sufficient to make 100%.

A variation that does not limit this patent comprising benzocaine (10%), vegetable glycerin (5%), sodium hydroxymethylglycinate (0.7%), aloe vera extract (0.2%), carbomer (0.7%), and deionized water in quantities sufficient to make 100%.

A second variation that does not limit this patent comprises aloe vera juice (10%), vegetable glycerin (5%), sodium hydroxymethylglycinate (0.7%), carbomer (0.7%), linalool (0.5%), and deionized water in quantities sufficient to make 100%.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

1. A sterile ultrasound gel stored or sold in a 50 ml or larger container comprising the preservatives sodium hydroxymethylglycinate at a concentration of 1% or less and phenoxyethanol at a concentration of 1% or less.

2. The product in claim #1 that is stored or transported in an airless dispenser.

3. The product in claim #1 that contains 1% or less fungicidal essential oil or component of fungicidal essential oil.

4. The product in claim #1 that contains 1% or less anesthetic essential oil or component of an anesthetic essential oil.

5. The product in claim #1 that comprises the humectant aloe vera juice at a concentration of 10% or greater or aloe vera concentrate at a concentration between 0.1% and 1%.

6. A sterile ultrasound gel stored or sold in a 50 ml or larger container comprising the preservatives sodium hydroxymethylglycinate at a concentration of 1% or less and a -caine fungistatic anesthetic at a concentration of 10% or less.

7. The product in claim #6 that is stored or transported in an airless dispenser.

8. The product in claim #6 that contains 1% or less fungicidal essential oil or component of fungicidal essential oil.

9. The product in claim #6 that contains 1% or less anesthetic essential oil or component of an anesthetic essential oil.

10. The product in claim #6 that comprises the humectant aloe vera juice at a concentration of 10% or greater or aloe vera concentrate at a concentration between 0.1% and 1%.

11. A sterile ultrasound gel stored or sold in a 50 ml or larger container comprising the preservatives sodium hydroxymethylglycinate at a concentration of 1% or less and a component of a fungicidal essential oil at a concentration of 1% or less.

12. The product in claim #11 that is stored or transported in an airless dispenser.

13. The product in claim #1 that contains 1% or less anesthetic essential oil or component of an anesthetic essential oil.

14. The product in claim #11 that comprises the humectant aloe vera juice at a concentration of 10% or greater or aloe vera concentrate at a concentration between 0.1% and 1%.
